(19) United States
(12) Patent Application Publication
(10) Pub. No.: US 2006/0025757 A1
(43) Pub. Date: Feb. 2, 2006

(54) Multielectrode electrosurgical instrument

(76) Inventor: Warren P. Heim, Boulder, CO (US)

Correspondence Address: MARSH, FISCHMANN & BREYFOGLE LLP, 3151 SOUTH VAUGHN WAY, SUITE 411, AURORA, CO, 80014, US

(21) Appl. No.: 11/185,668

(22) Filed: Jul. 20, 2005

Related U.S. Application Data
(60) Provisional application No. 60/589,508, filed on Jul. 20, 2004.

(51) Int. Cl. A61B 18/04

(52) U.S. Cl. 606/32

(57) ABSTRACT

An improved electrosurgical instrument and method is disclosed for simplifying making incisions and other treatments using electrosurgery. The electrosurgical instrument comprises a body having more than two electrodes with at least two electrodes having alternating current power supplied to them such that they comprise a bipolar alternating current configuration and employ a means other than electrode spacing, composition, or geometry for reducing or preventing accumulation of eschar that would otherwise form a short circuit current path and interfere with obtaining a predetermined surgical effect. In one aspect, such means for reducing or preventing eschar accumulation consists of at least one other electrode having a direct current voltage between it and at least one of the two electrodes forming the alternating current bipolar configuration. In another aspect of the invention two or more pairs of alternating current electrodes comprising bipolar electrodes are powered with alternating current having a nonzero RMS voltage sufficient to at least reduce eschar accumulations on one electrode or induce electrolysis of at least one component of a medium surrounding at least one pair of bipolar electrodes. The electrodes are separated from each other using electrically insulating materials such that electric current does not flow between at least two of the bipolar alternating current electrodes unless they contact at least one other electrically conductive medium, such as patient tissue or a medium comprising at least in part a solid, liquid, gas, or ionized component that allows electric current to flow between electrodes. In the aspect where at least one electrode is powered by direct current the electrodes are configured such that electrical current does not flow between at least one of the bipolar alternating current electrodes and at least one of the direct current electrodes unless one or more media, such as patient tissue or a medium comprising at least in part a solid, liquid, gas, or ionized component that allows electric current to flow between electrodes, are contacting or adjacent to the electrodes having a direct current voltage difference between them. The assembly comprised of the electrodes and the separating insulating materials may also employ one or more means to reduce the current flowing between them that does not produce a desired predetermined surgical effect with one aspect of such means being using an outer insulating coating configured such that one or more portions of at least one of the bipolar alternating current electrodes are insulated while leaving exposed other portions of such insulated electrodes so that they are capable of being in electrical communication with tissue or at least one material in electrical communication with tissue.

### CROSS-REFERENCE TO RELATED APPLICATIONS

This application claims priority under 35 U.S.C. §119 to prior U.S. Provisional Patent Application Ser. No. 60/589,508, filed Jul. 20, 2004, the entirety of which is hereby incorporated by reference.

### FIELD OF THE INVENTION

The present invention relates to surgical methods and apparatus for applying electrosurgical power to a tissue site to achieve a predetermined surgical effect, and more particularly, to an improved electrosurgical instrument and method to achieve such effect without using a return electrode pad separate from the device used to produce the predetermined surgical effect.

### BACKGROUND OF THE INVENTION

The potential applications and recognized advantages of employing electrical energy in surgical procedures continue to increase. In particular, for example, electrosurgical techniques are now being widely employed to provide significant localized surgical advantages in open, laparoscopic, and arthroscopic applications, relative to surgical approaches that use mechanical cutting such as scalpels.

Electrosurgical techniques typically entail the use of a hand-held instrument, or pencil, that transfers alternating current electrical power operating at radio frequency (RF) to tissue at the surgical site, a source of RF electrical power, and an electrical return path device, commonly in the form of a return electrode pad attached to the patient away from the surgical site (i.e., a monopolar system configuration) or a smaller return electrode positionable in bodily contact at or immediately adjacent to the surgical site (i.e., a bipolar system configuration). The time-varying voltage produced by the RF electrical power source yields a predetermined electrosurgical effect, such as tissue cutting or coagulation.

Despite numerous advances in the field, currently employed electrosurgical techniques for making incisions using blades are limited to monopolar electrosurgery, i.e. they use return pads. Bipolar electrosurgical devices exist in the forms of at least forceps and scissors, but successful application of bipolar electrosurgery for making incisions with blades has not occurred. Bipolar electrosurgery is widely recognized as providing inherently better patient safety than monopolar electrosurgery because the electrical current travels only a very short distance through the patient, compared to the much longer travel path from instrument to return pad that occurs with monopolar electrosurgery.

All devices that may be used to produce a predetermined surgical effect by applying RF power to tissue, such as causing a partial or complete separation of one or more tissue structures or types, including, but not limited to making electrosurgical incisions, or that cause partial or complete removal of one or more parts of a tissue, or that change the structure of tissue, such by at least partially denaturing or decomposing tissue, will be referred to as electrosurgical blades regardless of their size, shape, or other properties. Although they may have various forms, all sources of RF power used to power blades will be referred to as electrosurgical units and abbreviated by ESU. Monopolar electrosurgical blades connect to an ESU using a wire and a separate return pad is connected to the ESU with another wire. Bipolar electrosurgical blades connect a set of one or more active electrodes to the ESU with one or more wires and connect another set of one or more return electrodes to the ESU with one or more other wires.

Prior to the present invention, bipolar blades suffered from requiring that the electrodes be close enough together so that current would reliably pass into tissue but not be so close together as to allow short circuiting to occur though a bridge of conducing material, such as carbonaceous material formed from thermally decomposed tissue products. Such deposits of thermally decomposed tissue products are called eschar. Eschar readily forms in the high temperature environment local to electrosurgical blades. When electrodes are placed far enough apart to prevent short circuiting by eschar it becomes difficult to ensure that both active and return electrodes contact tissue. When the electrodes are close enough to ensure that both active and return electrodes contact tissue the rapid formation of short circuiting bridges ensues.

Prior art for bipolar electrosurgery blades have replaced the return pad with one or more electrodes on the blade itself. The additional electrode(s) are connected to the ESU using a wire. The electrical path is generally described as coming from the ESU, to one electrode on the blade, through patient tissue, into the other electrode on the blade, and then back to the ESU. All of the prior art for bipolar blades has two or more electrodes, all of which are connected to the ESU such that they all experience the same voltage differences with such voltage differences either being direct current or alternating current and never a combination of the two types of electrical energy. For example, an early U.S. Pat. No. 164,184 for a bipolar electrosurgical device describes using a pair of conductors spirally wound onto a rubber probe body in which the conductors are embedded. The device is not used to make incisions and uses direct current supplied from a battery to apply the same voltage difference to all electrodes. A bipolar electrosurgical device described in U.S. Pat. No. 1,983,669 has a pair of conductors twisted around an insulator that is powered by high frequency (i.e., alternating current) energy. U.S. Pat. No. 4,011,872 shows an electrosurgical device using one conductor connected to a high frequency energy source and formed of three or four electrodes.

The electrodes may take on a variety of configurations, as described using the following exemplary prior art. In U.S. Pat. No. 3,970,088, U.S. Pat. No. 3,987,795, and U.S. Pat. No. 4,043,342, all by Morrison, electrode configurations are disclosed wherein the surface areas of the active and return electrodes are substantially different. The Morrison patents disclose using a porous material surrounding electrodes to enhance stable startup. The Morrison patents further disclose using multiple electrodes in which all of the electrodes are connected to the ESU such that the RF power is applied to all of the electrodes. U.S. Pat. No. 4,202,337 and U.S. Pat. No. 4,228,800 disclose bipolar blade configurations with split electrodes in which all of the electrodes are connected to the ESU such that RF power is applied to all of the electrodes. The '337 and '800 patents further disclose bipolar blades that insert into a handle that has electrical contacts that provide electrical connections to the ESU such that a pair of side electrodes are shorted together and act as the return electrode with a center electrode acting as the active electrode. U.S. Pat. No. 4,232,676 discloses pairs of electrodes in which the voltage applied may be either direct current or alternating current but in either case the voltage applied to all of the electrodes is the same. U.S. Pat. No. 4,706,667 discloses a pair of return electrodes flanking a cutting electrode. U.S. Patent Application Publication No. 20030130658 discloses multiple electrodes having dissimilar materials in which RF power is applied to all of the electrodes.

Notably absent from prior art are means for preventing short circuiting from tissue fragments or tissue decomposition products accumulating on blades when electrodes are placed close together to ensure reliable contacts between electrodes and tissue. The need remains for a bipolar blade that reliably contacts tissue with multiple electrodes and that inhibits short circuiting by eschar.

### SUMMARY OF THE INVENTION

Accordingly, a primary objective of the present invention is to provide an apparatus and method for use in electrosurgery that results in reliable electrode contact with tissue and inhibits short circuiting.

Another objective of the present invention is to provide an apparatus and method for use in electrosurgery that yields less eschar accumulation on the electrosurgical instrument utilized.

An additional objective of the present invention is to provide an apparatus and method for use in electrosurgery that provides for reduced charring along an electrosurgical incision.

An additional objective of the present invention is to provide an apparatus and method for use in which the amount of electrosurgical smoke produced is reduced.

Yet another objective is to realize one or more of the foregoing objectives in a manner which does not significantly impact space or cost requirements, and which maintains and potentially enhances the effectiveness of electrosurgical procedures.

In addressing these objectives, the present inventors have recognized that applying a direct current between the electrodes of a bipolar electrosurgical blade reduces or prevents the formation of short circuits, even when the electrodes in blades are close together. The present inventors have further recognized that the propensity for such short circuiting to occur can be reduced by limiting the amount of exposed electrode surface area. The present inventors have yet further recognized that such application of direct current between electrodes and limiting of electrode surface areas are mutually beneficial and complement each other.

More generally in this regard, energy discharge from electrosurgical instruments may be in the form of electrical energy and/or thermal energy. Electrical energy is transferred whenever the electrical resistance of a region between an electrosurgical instrument and tissue can be broken down by the voltage of the electrosurgical power. Thermal energy is transferred when thermal energy that has accumulated in the electrosurgical instrument overcomes the thermal resistance between the instrument and the tissue (i.e. due to temperature differences therebetween). Such transfers of electrosurgical energy may occur at portions of the electrosurgical instrument that lead to a desired surgical effect, such as forming an incision. Such portions of the instrument are called functional areas. All other areas of the electrosurgical instrument are nonfunctional.

The discharge of energy into tissue causes many effects, including decomposing the tissue into smaller parts having the same structure or different structures than existed prior to the discharge of energy. The collection of processes that break down tissues during electrosurgery will be called electrosurgical tissue decomposition processes. Although the mechanisms of electrosurgical tissue decomposition processes are not well understood, at least part of this process is believed to be tissue pyrolysis. Electrosurgical tissue decomposition processes lead to the formation of substances that adhere to electrosurgical blades. The combination of substances are at least somewhat electrically conductive at the voltages and frequencies of electrical power employed during electrosurgery. The combination of substances typically take the form of a carbon-rich material called eschar. When bipolar blades are used eschar tends to start to form on one electrode or another. The deposit then grows in thickness as it propagates from that electrode, increasing the electrical impedance at that electrode from what it would be absent the eschar deposit. As the eschar deposit grows it can span the gap between active and return electrodes in bipolar devices, leading to a short circuit current path for the RF power that reduces or prevents power transfer to tissue, thus interfering with or preventing the desired surgical effect from occurring.

In short, the present inventors have recognized that a means is needed to prevent the formation or accumulation of the short circuits from materials formed by electrosurgical tissue decomposition processes. The present invention comprises an electrosurgical instrument that includes a multiplicity of electrodes with at least one active and at least one return electrode. In a system context, the electrodes of the electrosurgical instrument have not only alternating current flowing but also direct current flowing between at least one active electrode or at least one return electrode and another electrode. Such direct current reduces or prevents the formation and accumulation of electrosurgical tissue decomposition products on electrodes. The mechanisms by which direct current reduces or prevents eschar accumulation are not precisely known but are believed to include effects caused by electrolysis of water and shifts in chemical reactions. Electrodes having a more negative voltage are believed to accumulate small amounts of hydrogen in a layer believed to restrict eschar accumulation. The negative charge is also believed to inhibit dehydrogenation reactions that would otherwise occur at the temperatures that exist during electrosurgery, thus inhibiting the formation of at least some of the carbon-rich constituents that comprise eschar.

The method of reducing eschar on bipolar blade electrodes by applying direct current may be applied when other means are employed to reduce unnecessary/undesired electrical discharge during electrosurgical procedures. Such reduction(s) reduce the amount of direct current required to reduce or prevent eschar accumulations and are achieved via enhanced localization of electrical power transmission to a tissue site. More particularly, the present invention markedly reduces electrical discharge from both functional and nonfunctional areas of an electrosurgical instrument by insulating either or both functional and nonfunctional areas. The amount of direct current required to reduce or prevent eschar accumulation is reduced when one or means are employed to reduce the local heating that promotes eschar formation. Such means for reducing local heating include providing for an effective level of heat removal away from functional portions of an electrosurgical instrument and/or by otherwise enhancing the localized delivery of an electrosurgical signal to a tissue site such as by reducing the exposed areas of either or both functional and nonfunctional areas by using thermal insulation.

The present invention comprises an electrosurgical instrument that includes a multiplicity of electrodes for carrying electrosurgical power in which the electrodes are electrically isolated from each other and provide for being connected to an ESU in an overall system such that at least one active electrode and at least one return electrode exist, thus forming at least one set of bipolar electrodes. In one aspect of the present invention direct current voltage may be applied across this pair of electrodes to reduce or prevent formation of electrosurgical tissue decomposition products (ETDPs) such as eschar. The electrode with the negative DC voltage will have little or no accumulation of ETDPs. However, the electrode with the positive DC voltage will tend to accumulate ETDPs. A further aspect of the present invention is to include at least one electrode in the system that is not directly connected to the RF power coming from the ESU. An electrode not powered by the ESU does not directly produce the predetermined surgical effect and any such electrodes are called passive electrodes herein. All passive electrodes are connected to one pole of a DC power source and the bipolar electrodes are connected to the other pole of the DC power source. Typically, the passive electrodes would be connected to the positive pole and the bipolar electrodes would be connected to the negative pole of the DC power source. Therefore, both of the bipolar electrodes are connected to RF power, which produces the predetermined surgical effect and tends to produce ETDPs, and to a DC power source, while the passive electrodes are connected only to DC. In the typical system configuration the negative DC on the bipolar electrodes prevents or reduces accumulations of ETDPs and the absence of RF power on the passive electrodes prevents or reduces accumulations of ETDPs on them.

In a typical electrosurgical instrument designed to make incisions there would be one pair of bipolar electrodes and one passive electrode. The regions near bipolar electrodes have temperatures that tend to promote eschar formation, but the negative DC current inhibits or prevents eschar accumulation. The passive electrodes are not powered by the ESU so the regions around them do not have the conditions that promote eschar formation.

In an electrosurgical instrument used to produce an electrosurgical effect on tissue in an environment where the electrodes are surrounded by a medium that provides electrical communication between at least one of the bipolar electrodes and tissue, an example of such a medium being an electrically conductive liquid containing substantial amounts of water, one or more pairs of bipolar electrodes may be employed, with or without the presence of one or more passive electrodes. In this second instance the bipolar electrodes of the system would have RF power applied to them that has a voltage bias that leads to a nonzero root mean square (RMS) voltage that is adequate to electrolyze water local to the electrodes. When one or more passive electrodes are used the connections to DC power would be as previously described and electrolysis would also occur. The electrolysis of water produces at least a partial covering of gas bubbles around enough electrodes to create a sufficient impedance between the bipolar electrodes for an ESU to supply power adequate to produce the desired electrosurgical effect.

When one or more passive electrodes are used the bipolar electrodes are both connected to the same pole of a DC power source. To prevent this common connection from shorting the active and return electrodes one or more electronic AC blocking components that allow DC current to flow while inhibiting passage of alternating current are put in series with the connections from the DC power source to the bipolar electrodes. Typically the components would be inductors sized to produce substantial impedance, such as over about 500 ohms, to the RF power produced by the ESU while producing acceptably small DC resistance, such as less than about 100 ohms. The direct current voltage difference between one or more passive electrodes and one or more of the bipolar electrodes needs to be adequate to at least inhibit eschar accumulation while not producing too much electrolysis, such as by being at least about 0.5 volts and less than about 100 volts. Relatedly, insulating material may be interposed and interconnected between at least the two bipolar alternating current electrodes to define an electrosurgical blade. Such electrical insulating material preferably has a dielectric withstand strength of at least 50 volts and may comprise either a single layer or multiple layers with one or more other electrodes interposed between insulation layers.

In one aspect of the invention three electrodes that are substantially colinear over at least one dimension are used with at least part of the electrodes oriented parallel to each other with all of the electrodes separated from and physically interconnected to the other electrodes by one or more electrical insulating materials to define an electrosurgical blade. The electrosurgical blade may be configured so that at least part of each electrode may contact tissue or an electrically conductive substance in contact with tissue, with two of the electrodes being bipolar electrodes with an alternative current voltage applied to them and the remaining electrode having a direct current voltage difference between it and the bipolar electrodes.

In another aspect of the invention an outer insulating layer consisting of one or more materials to reduce thermal/electrical discharge from non-functional portions of the electrodes may be provided to surround at least a portion of the bipolar electrodes. For example, an outer insulating layer having a thermal conductance of about 1.2 W/cm² ° K and a dielectric withstand strength of at least about 50 volts may be employed. Such insulating layer may advantageously comprise one or more materials with pores that have been sealed with a sealing material so as to prevent biological materials from entering the pores. with Such sealing material preferably contains a colloidal silicate material and may further comprise one or more hydrolyzable materials that in combination form a thermally insulative substance that by itself is essentially hydrophobic and does not allow biologic material to penetrate its surface.

In another aspect of the invention, one or more of the electrodes are metal with the electrodes provided to have a thermal conductivity of at least about 0.35 W/cm ° K, and may advantageously comprise a metal selected from the group: gold, silver, aluminum, copper, tantalum, tungsten, columbium, and molybdenum. In a related aspect of the invention one or more of the electrodes may be coated or plated with a substance or element that imparts resistance to oxidation such as a plating of gold or silver. In yet a further related aspect, the electrodes may include an intermediate layer that defines a peripheral edge portion of reduced cross-section (e.g., about 0.001 inches thick or less) for electrosurgical power or direct current power transmission. Such intermediate layer may comprise a metal having a melting point of at least about 2600° F. Heat sink means may be included in various embodiments to establish a thermal gradient away from functional portions of the instrument (i.e., by removing heat from the electrodes). In one embodiment, the heat sink means may comprise a phase change material that changes from a first phase to a second phase upon absorption of thermal energy from the electrodes.

In another aspect of the present invention an electrosurgical blade is provided in which the electrodes are spaced apart using one or more types of electrically insulating particles, such as polymeric, glass, or ceramic beads, that have maximum cross dimensions approximately equal to the distance desired for spacing the electrodes from each other. In this regard, the spacing particles may be included as part of the above-noted electrical insulating material provided between the electrodes. In turn, the particles may be at least partially in contact with at least one additional material of the electrical insulating material that bonds to the electrodes of the electrosurgical blade.

Additional aspects and advantages of the present invention will be apparent to those skilled in the art upon consideration of further description that follows.

### BRIEF DESCRIPTION OF THE DRAWINGS

FIG. 1 portrays a system schematic with a general multielectrode blade having active, passive, and return electrodes.

FIG. 2 portrays a system schematic with a general multielectrode blade having active, passive, and return electrodes with an activation switch.

FIG. 3 portrays a system schematic with a multielectrode blade having active, passive, and return electrodes with DC power derived from the RF power.

FIG. 4 portrays a system schematic with a multielectrode blade having active and return electrodes with DC power derived from the RF power.

FIG. 5 portrays a system schematic with a multielectrode blade having active and return electrodes with DC power derived from the RF power with an activation switch.

FIG. 6 illustrates a side view of an electrosurgical instrument having an electrode blade.

FIG. 7 portrays a cross section of a multielectrode blade having active, passive, and return electrodes with a substantially flat contact face.

FIG. 8 portrays a cross section of a multielectrode blade having active, passive, and return electrodes with a convex contact face.

FIG. 9 portrays a cross section of a multielectrode blade having active, passive, and return electrodes with a convex contact face with electrode edges exposed.

FIG. 10 portrays a perspective view of a portion of a multielectrode blade with insulation cut away to show active, passive, and return electrodes tapered to a convex contact face having electrode edges exposed.

FIG. 11 portrays a cross section of a multielectrode blade having active, passive, and return electrodes with a convex contact face having electrode edges exposed and an adjacent concave surface.

### DETAILED DESCRIPTION OF THE INVENTION

The present invention is for a multielectrode electrosurgical instrument and related system and method that employ a means for reducing or preventing eschar accumulations on or between electrodes by a means other than the spacing between electrodes, geometry of electrodes, or composition of electrodes. Such means of reducing or preventing eschar accumulations on or between electrodes may require or be augmented by electrode spacing, geometry, or composition. The present invention applies to instruments in which at least one pair of electrically isolated electrodes are mechanically connected such that their spacing is limited to a predetermined range (such range possibly being a fixed distance) and electrically connected to an ESU such that RF current will flow between the electrodes when they contact an electrically conductive medium such as tissue or an electrically conductive liquid or vapor. These electrodes are bipolar electrodes and any device having one or more sets of bipolar electrodes is a bipolar instrument. All bipolar instruments, regardless of their intended purpose, design, shape, geometry, configuration, materials, or other aspects are referred to as electrosurgical blades.

The preferred embodiment of the means for reducing or preventing eschar accumulations on or between bipolar electrodes is to have direct current flow through at least one of the bipolar electrodes with at least part of the current flow passing through tissue or passing through at least one electrically conductive medium in electrical communication with at least one of the electrodes. In one embodiment the direct current flows between both of the electrodes of a pair bipolar electrodes with at least part of the current flowing through tissue or passing through at least one electrically conductive medium in electrical communication with at least one of the electrodes. An even more preferred embodiment is to have at least one pair of bipolar electrodes and for at least one passive electrode (an electrode not be powered by an ESU) and for direct current to flow between said passive electrode and at least one of the bipolar electrodes with said direct current at least in part flowing through tissue or passing through at least one electrically conductive medium in electrical communication with at least one of the electrodes.

Bipolar or passive electrodes may be any shape or shapes such as, but not limited to, being substantially flat, having one or more curves, being shaped as closed curves such as rings or hoops, being shaped as nonclosed curves such as semicircles or crescents, being planar, being nonplanar such as curved spatulas, having bends or curves such as hooks, encompassing volumes such as cups or cylindrical volumes, being substantially blunt, having one or more regions that taper from one thickness to a lesser thickness, being solid such as spheres or balls, having opposing faces such as forceps or scissors, and having one or more openings such as holes, meshes, pores, or coils.

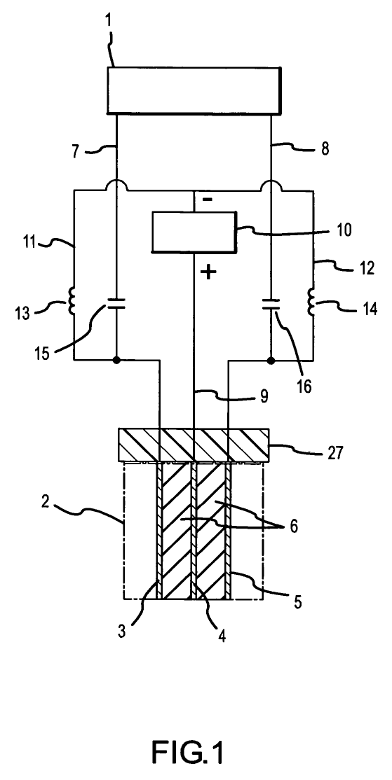

FIG. 1 illustrates the preferred embodiment in which a passive electrode is used. ESU 1 supplies power to multielectrode blade 2. Multielectrode blade 2 consists of one or more active electrodes 3, one or more passive electrodes 4, one or more return electrodes 5, with the electrodes insulated from each other by interior insulation 6. Recognize that ESU 1 supplies alternating current power so that the flow of electric current between active electrodes 3 and return electrodes 4 periodically reverses as the voltage output of ESU 1 changes. Multielectrode blade 2 may be without insulation other than that separating the electrodes or additional insulation may surround the electrodes. These aspects of the invention are described later.

Active electrodes 3 may be one or more electrically conductive elements and whenever referred to in the singular case are understood to also include the use of a multiplicity of electrodes connected electrically to have substantially the same power source or power sources. Similarly, passive electrodes 4 may be one or more electrically conductive elements and whenever referred to in the singular case are understood to also include the use of a multiplicity of electrodes connected electrically to have substantially the same power source or power sources. Also similarly, return electrodes 5 may be one or more electrically conductive elements and whenever referred to in the singular case are understood to also include the use of a multiplicity of electrodes connected electrically to have substantially the same power source or power sources.

Power from ESU 1 to multielectrode blade 2 is conveyed via supply conductive element 7, which is preferably an insulated metal conductor for at least part of its length and terminates into handle 27 that holds multielectrode blade 2 in a manner that conveys power to active electrode 3 and that is convenient for having multielectrode blade 2 contact patient tissues. The electrical circuit for power from the ESU 1 to multielectrode blade 2 is completed via return conductive element 8, which is preferably an insulated metal conductor for at least part of its length and terminates into handle 27 that holds multielectrode blade 2 in a manner that conveys power from return electrode 5 to return conductive element 8.

Passive electrode 4 is powered by passive conductive element 9, which is preferably an insulated metal conductor for at least part of its length and terminates into handle 27 that holds multielectrode blade 2 in a manner that conveys power to passive electrode 3.

DC power supply 10 supplies power to passive electrode 4 via passive conductive element 9, preferably with the positive DC voltage being supplied to passive electrode 4. DC power supply 10 provides power to active electrode 4 via DC conductive element 11. DC power supply 10 provides power to return electrode 6 via DC conductive element 12.

One or more RF current impedance elements 13 and 14 are in DC conductive elements 11 and 12 so that supply conductive element 7 and return conductive element 8 are kept substantially isolated from each other and short circuit and to substantially isolate passive electrode 4 and DC power supply 10 from being in RF current paths parallel to supply conductive element 7 or return conductive element 8. RF current impedance elements 13 and 14 are preferably inductive elements providing at least about 500 ohms impedance at the output frequency of ESU 1 and more preferably providing at least about 1000 ohms impedance at the output frequency of ESU 1 and still more preferably providing at least about 5000 ohms impedance at the output frequency of ESU 1. RF current impedance elements 13 and 14 preferably are at least about 50 microhenries and more preferably at least about 1000 microhenries and still more preferably about 10,000 microhenries. RF current impedance elements 13 and 14 need to convey DC power and preferably are capable of carrying at least about 5 milliamperes and more preferably at least 50 milliamperes and preferably have a DC resistance of less than about 100 ohms and more preferably of less than about 50 ohms and still more preferably less than about 20 ohms.

DC power supply 10 preferably provides voltage in the range of about 0.5 volt to 100 volts and more preferably in the range of about 2.5 volts to 50 volts and still more preferably in the range of about 5 volts to 20 volts. DC power supply 10 preferably provides current in the range of about 0.0100 milliamperes to 1 ampere and more preferably in the range of about 10 milliamperes to about 0.1 ampere.

ESU 1 is isolated from DC power by the presence of one or more DC blocking elements 15 and 16. DC blocking elements are preferably capacitors having a low equivalent series resistance (ESR) at the frequency of the power from ESU 1 and having an impedance of less than about 500 ohms and preferably less than about 100 ohms and still more preferably less than about 50 ohms and even yet more preferably of less than about 10 ohms at the output frequency of ESU 1. In some cases DC blocking element 15 may be omitted and DC current flow being blocked by DC blocking element 16 is adequate.

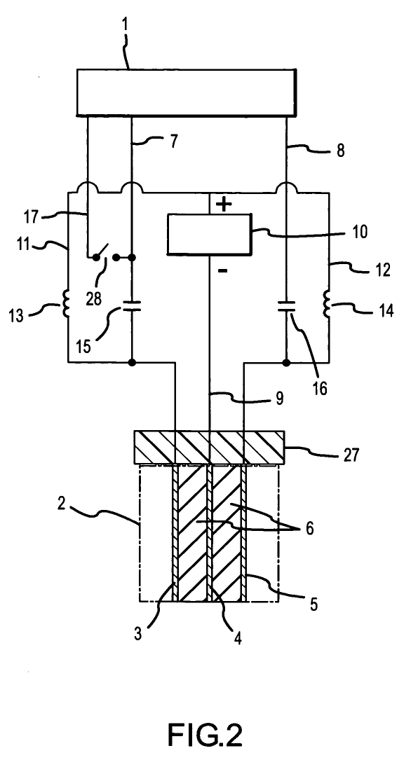

Users control when ESUs supply power by using either a footswitch or a switch in a handle that holds blades. When the switch is in the handle it is common for one or more signal wires to come from the ESU to the handle and for the supply conductive element 7 to be part of the signal path. As is known to those skilled in the art of ESU design, the RF power supply and the signal path are isolated and separated in the ESU and commonly the control signal is a DC signal that uses the supply conductive element 7. To not interfere with this control strategy the DC blocking element 15 must be located to not prevent the control signal from reaching ESU 1. FIG. 2 illustrates the same schematic as FIG. 1 with the addition of control signal conductive element 17 and control switch 18. Preferably, control signal conductive element 17 and control switch 28 are in the handle 27, although they are not shown that way in FIG. 2. DC blocking element 15 is placed so that it is not in series with control switch 28 and ESU 1. Preferably, DC blocking element 15 is located in handle 27.

DC power supply 10 may take on any form that provides the proper voltage and current. In one embodiment it may be one or more batteries. In another embodiment it may be an external power supply powered from a power cord connected to AC line power from a wall outlet or power from a connection in ESU 1. The preferred embodiment obtains DC power from the RF power supplied by ESU 1. In the preferred embodiment DC power supply 10 contains one or more active components, such as diodes or other rectifying elements, and is connected to the RF output of ESU 1 and converts part of the RF output from ESU 1 into DC power.

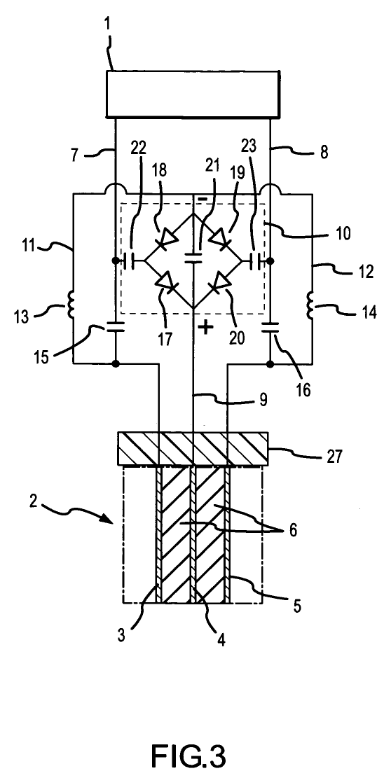

FIG. 3 illustrates a bridge circuit that produces DC power from the RF power supplied by ESU 1. DC power supply 10 contains four rectifying elements, 17, 18, 19, and 20, configured in a bridge configuration. A voltage control system 21 controls the output voltage. Voltage control system 21 is illustrated as a capacitor but may consist of one or more active elements to further control voltage. The voltage control system may consist of a capacitor to reduce the magnitude of the voltage deviations. Preferably, the voltage control system will include a means for controlling the maximum output voltage, such as by using a zener diode in series with a resistive load. One or more RF voltage reduction elements, 22 and 23, are used to drop the voltage output by ESU 1 to produce a DC output voltage in the range desired. The presence of one or more RF voltage reduction elements, 22 and 23, reduces the power dissipation requirements that may be imposed on voltage control system 21. Rectifying elements, 17, 18, 19, and 20 are preferably diodes and may be of any type that has a reverse recovery time compatible with frequencies of at least 20 kHz and more preferably of at least 100 kHz and even more preferably of at least 200 kHz and still more preferably of at least 500 kHz to be compatible with most of the ESUs being used, and finally compatible with at least 2 MHz to be compatible with almost all ESUs being used. Rectifying elements, 17, 18, 19, and 20 need to withstand the voltages output by the ESU and the RF voltage reduction elements, 22 and 23 allow the use of a range of diodes, such as Schottky diodes, that can withstand preferably at least 500 volts and more preferably at least 1000 volts.

One or more of the elements of DC power supply 10, RF current impedance elements 13 and 14, and DC blocking elements 15 and 16 may be incorporated into the ESU 1, incorporated into an adapter that connects to ESU 1, incorporated into plugs and connectors used to connect supply conductive element 7 and return conductive element 8 to ESU 1 (these plugs and connectors are not shown in FIGS. 1, 2, or 3), or may be incorporated into the handle 27.

Typically, connections are made to ESUs with a plug that connects supply conductive element 7 to a power supply connector on the ESU and with another plug that connects return conductive element 8 with a return connector on the ESU. In the preferred embodiment of the present invention the elements of DC power supply 10, RF current impedance elements 13 and 14, and at least one of the DC blocking elements 15 or 16 are housed in a plug that connects the supply conductive element 7 to the ESU and that has a wire that passes from it to a plug that connects to a return connector on the ESU. Such an embodiment may either be reusable or may be a single use sterile disposable.

FIGS. 1, 2, and 3 illustrate the passive electrode being between the active and return electrodes. This arrangement is not required. Passive electrodes may be anywhere that allows them to be in electrical communication with the active electrodes. Passive electrodes do not need to be mechanically connected to the device of which the active and passive electrodes are a part. For example, one or more passive electrodes could attach to the patient in the form of one or more electrode pads and connect to DC power supply 10 using a wire. Preferably, the passive electrodes are mechanically connected to the device of which the active and passive electrodes are a part.

The electrodes may be any shape, size, or arrangement that leads to a configuration and composition suitable for a particular application. For example, an arthroscopic ablation instrument used in a submerged electrically conductive liquid may be configured with multiple active and return electrodes with suitable shapes, such as in the form of linear or curved edges or pins, close together at the end of a shaft and a single passive electrode could be spaced back away from end of the shaft and be in the form of a ring around the shaft. All of the electrodes would be surrounded by electrically conductive liquid and, thus, be in electrical communication with the liquid. In another arrangement, a split ring that forms a bipolar pair could have inlaid a passive electrode.

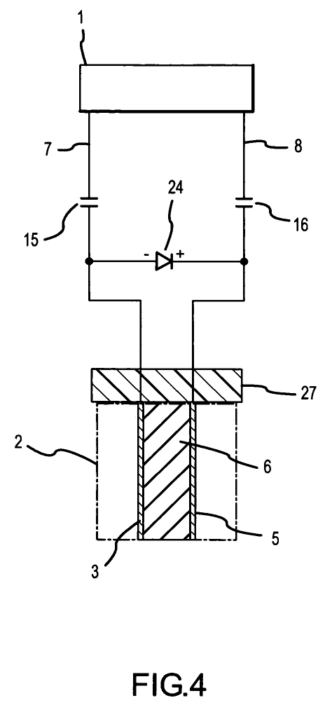

FIG. 4 illustrates an embodiment that uses fewer components than those illustrated in FIGS. 1-3. Direct current is supplied directly between active electrode 3 and return electrode 5. No passive electrode is used. Any direct current power source may be used when direct current is supplied directly between the bipolar electrodes. The preferred embodiment uses RF power supplied by the ESU and one or more rectifying elements. FIG. 4 illustrates using rectifying element 24 to produce a DC voltage. Contrary to common design practice for rectifiers such as diodes, rectifying element 24 preferably has a reverse recovery time less than the period of the AC power supplied. Preferably the reverse recovery time of rectifying element 24 is between about 0.05 and 0.5 the period of the AC power supplied and more preferably is between about 0.1 and 0.25 the period of the AC power supplied. Using such reverse recovery times leads to substantial reverse current flow through the rectifying diode before it starts to inhibit backwards current flow. This slow response leads to a substantially lower direct current voltage being applied across active electrode 3 and return electrode 4 than would otherwise occur using common design practice. Preferably, diodes rated as standard or fast recovery are used. Preferably, diodes with a voltage withstand of at least 300 volts and more preferably at least 1000 volts are used.

DC blocking element 15 can interfere with passage of control signals that may need to pass between one or more switches in handle 27 and ESU 1. FIG. 5 illustrates a more preferred embodiment that does not include a DC blocking element in the supply conductive element 7. Control signal conductive element 17 and control switch 28 are illustrated to show that control switch 28 can be located anywhere. Preferably, it is located in handle 27.

In configurations without passive electrodes the electrodes may be any shape, size, or arrangement that leads to an arrangement suitable for a particular application. For example, an arthroscopic ablation instrument used in a submerged electrically conductive liquid may be configured with multiple active and return electrodes with suitable shapes, such as in the form of linear or curved edges or pins, close together at the end of a shaft. All of the electrodes would be surrounded by electrically conductive liquid and, thus, be in electrical communication with the liquid. In another arrangement, a split ring that forms a bipolar pair could have inlaid a passive electrode.

Alternatives to the illustrated preferred embodiments exist. For example, the embodiments of FIGS. 4 or 5 would tend to keep eschar from accumulating on active electrode 3 but not offer the same level of protection to return electrode 5. A passive electrode with a separate DC supply could be included that would cause DC current to pass between the passive electrode and the return electrode and reduce or prevent eschar accumulations on return electrode 5.

ESU 1 may have multiple RF supplies connected via a multiplicity supply and return conductive elements to a multiplicity of active and return electrodes that are not electrically connected and thus operating substantially independently of each other to provide multiple voltage waveforms, possibly with phase angles, frequencies, and voltages that differ from one another. DC power supply 10 may have multiple direct current power sources connected via a multiplicity of passive supply conductive elements to a multiplicity of passive electrodes or to a multiplicity of active or return electrodes that are isolated from one another from DC current supply.

Passive electrodes need to be close enough to the bipolar electrodes to allow DC current to flow between the passive electrodes and the bipolar electrodes. The passive electrodes preferably contact patient tissue within six feet of the bipolar electrodes, and more preferably would be contacting patient tissue within six inches, and still more preferably within one inch of the bipolar electrodes. For many blades, such as those used for incisions, it is preferable for the passive electrodes to be within about 0.5 inches and more preferably within about 0.1 inches and still more preferably within 0.010 inches of the bipolar electrodes. The closer spacing between the passive electrodes and the bipolar electrodes reduces the overall size of the instruments and reduces the amount of tissue through with which DC current passes.

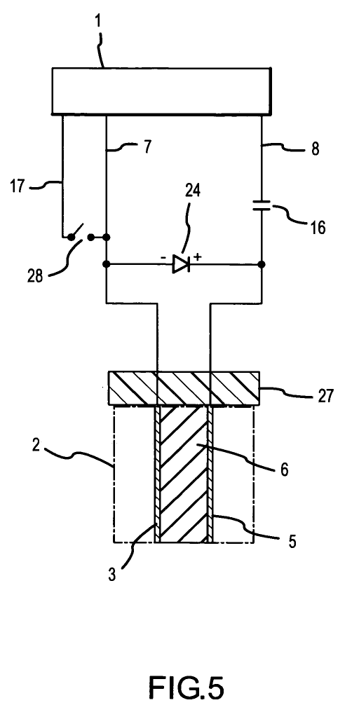

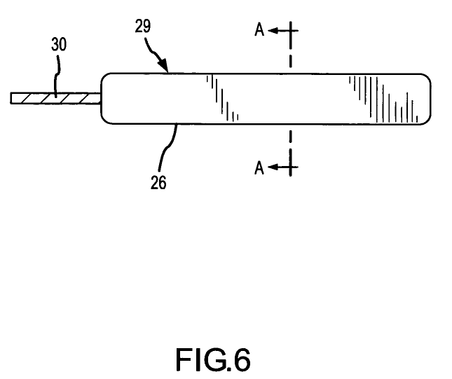

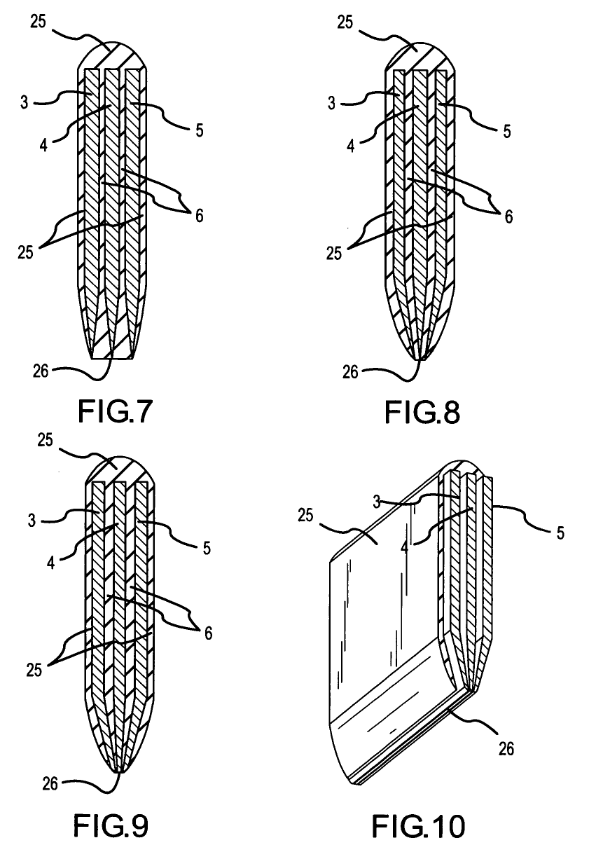

FIG. 6 illustrates an electrosurgical instrument configuration with blade 29 connected to shaft 30. Shaft 30 typically connects to a handle (not shown) and typically provides means in the form of one or more conductors for conveying electrical power to blade 29. The blade 29 includes a functional portion, or contact face 26 (e.g. a cutting edge), for contacting patient tissue. FIGS. 7, 8, 9, and 11 are cross sections of blade 29 in FIG. 6 when viewed through cross section AA.

FIGS. 7, 8, 9, 10, and 11 illustrate preferred configurations of multielectrode blades. To reduce DC current flow the preferred embodiment limits the amount of exposed electrodes surface area by extending interior insulation 6 over all of the interior surfaces of the electrodes except for the functional surfaces. To further reduce DC current flow the preferred embodiment employs outside insulation 25 to limit the electrode surface area exposed on the outside of the blades. These configurations show passive electrode 4 between active electrode 3 and return electrode 5. As described earlier, this arrangement is not required. However, when it is used it is particularly preferable is insulate the nonfunctional surfaces of active electrodes 3 and return electrodes 5. If, for example, the active electrode is between the passive and return electrodes the outer surface of the passive electrode does need to be insulated to reduce DC current flow when only the functional areas of active and return electrodes already have limited surface areas exposed.

FIG. 7 illustrates a configuration in which the electrodes taper to fine edges and the blade is shaped to present a substantially flat contact face 26 where only the sharp edges of the electrodes are exposed through the surface of the interior insulation 6 and outside insulation 25. FIG. 7 illustrates only one active electrode 3 and one return electrode 5, however multiple active and return electrodes could extend out in an alternating arrangement. Such arrangements would increase the size of the flat contact face 26 through which the electrodes emerge. These multiple electrode arrangements are preferred for applications where large surface areas are to undergo electrosurgical treatment, such as in arthroscopic tissue ablation procedures. For these applications an even more preferable embodiment has the electrodes emerging from the surface to form a rough surface that also mechanically abrades the surface of the tissue as it penetrates the tissue. For these applications it is preferable for the electrodes to protrude between 0.0001 and 0.5 inches and even more preferable for them to protrude 0.001 and 0.1 inches. For such applications it is preferable for a single passive electrode to be used and it is even more preferable for the passive electrode to be located away from the region where the active and return electrodes are located to maximize the amount surgical effect caused by the active and return electrode in a give surface area. The passive electrode is preferably attached to the shaft of the instrument within 0.5 inches of the location of the active and return electrodes and no restriction exists regarding minimum spacing between the passive electrode on the shaft and active or return electrodes on the working surface where the surgical effects occur.

For making incisions it is preferable for the width of the blade contacting tissue to be small to reduce drag. For making incisions it is further preferred to have small surface areas for functional areas and to also have small surface areas for nonfunctional areas near active and return electrodes to reduce the total exposed surface area where electrosurgical effects occur. Having small surface areas reduces the time that tissue is exposed to conditions that cause ETDPs and also reduces the residence time of ETDPs in the hot regions near the active and return electrodes. Long residence times tend to promote tissue decomposition and the ensuing formation of smoke, eschar, and collateral tissue damage. The preferred small exposed surface areas where electrosurgical effects occur reduce the formation of smoke, eschar, and tissue damage. The preferred embodiment for blades used for incisions is to taper a portion of the blade by tapering at least the outside insulation 25, as shown in FIG. 7 such that the narrowest part of the blade is the contact face 26 where the functional areas are located, which is the same areas where active electrode 3 and return electrode 5 are exposed. FIG. 8 illustrates the even more preferred embodiment in which the electrodes 3, 4 and 5, and insulation 6 and 25 are shaped to form a constantly curving strictly convex surface at the functional contact face 26. The strictly convex profile is preferred because it reduces the residence time of material in regions where smoke, eschar, and tissue damage occurs. FIG. 8 also illustrates the preferred embodiment in which electrodes are shaped to further taper the blade in order to reduce residence time. The strictly convex shape in the functional regions where the electrodes are exposed can be achieved without shaping the blades to accentuate the taper.

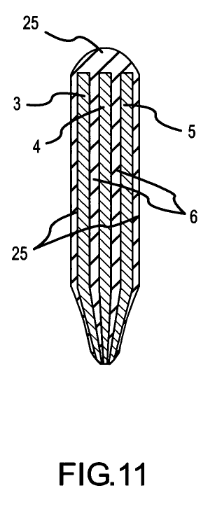

Even more preferred is for the tapered blade portion where the blade tapers toward the contact face 26 to be concave while keeping the contact face 26 where the electrodes are exposed strictly convex. FIG. 11 illustrates an embodiment that has a concave surface where the blade tapers and is convex where the electrodes are exposed. The strictly convex contact face 26 is most preferable where the electrodes contact the tissue and between the electrodes. The portions of the blades outside of the electrodes are most preferably tapered so that they are either flat or concave, as illustrated in FIG. 11.

For blades used for making incisions it is preferable for the blades to be thinner than about 0.5 inches and more preferable for them to be thinner than about 0.05 inches. When blades are too thick they impede the incision process and drag through the tissue. Metal electrodes preferably thinner than about 0.2 inches and more preferably thinner than about 0.1 inches and still more preferably thinner than about 0.02 inches should be used to produce blades with the desired thinness. Insulation thickness on the outside of the bipolar electrodes and the total insulation thickness between bipolar electrodes preferably thinner than about 0.2 inches and more preferably thinner than about 0.1 inches and still more preferably thinner than that about 0.02 inches should be used to produce blades with the desired thinness. The preferable spacing between electrodes is between about 0.001 and 0.2 inches, and more preferably between about 0.002 and 0.100 inches and most preferably between about 0.005 and 0.015 inches.

FIG. 9 and FIG. 10 illustrate a blade in which slightly more electrode metal than the very edge is exposed through the insulation. FIG. 10 illustrates an elongated blade in which the active electrode 3, passive electrode 4, and return electrode 5 extend along a dimension to form approximately coplanar surfaces and, consequently, has a contact face 26 in the form of a cutting edge that is substantially longer than it is wide. Such configurations are preferable for blades used to make incisions. Illustrated is a configuration in which the cutting edge of contact face 26 is approximately a straight line. The cutting edge could have other shapes, for example having one or more parts that have shapes that approximate part of an ellipse or circle instead of approximately a straight line.

If part of the edge is behind or covered by insulation then electrosurgical energy transfer is inhibited and accomplishing the corresponding desired predetermined electrosurgical effect is hindered. To provide reasonable manufacturing tolerance and not have part of the edges of electrodes exposed more than an extremely fine edge needs to be exposed. It is preferable that more than 90 percent of the active and return electrode edges along the functional surfaces be exposed and even more preferable that more than 95 percent of the active and return electrode edges be exposed along the functional surfaces and still more preferable that more than 99 percent of the active and return electrode edges be exposed along the functional surfaces. Furthermore, it is preferable to limit the DC current flow and residence time of tissues at the conditions that cause smoke, eschar, and tissue damage. Preferably the smallest dimensions (the widths) of the edges of the active and return electrodes are smaller than about 0.020 inches and even more preferably that the widths of the edges of the active and return electrodes are smaller than about 0.005 inches and still more preferable that the widths of the edges of the active and return electrodes are smaller than about 0.001 inches and still more preferable that the widths of the edges of the active and return electrodes are between about 0.00001 and 0.001 inches.

FIG. 10 illustrates the active electrode 3, passive electrode 4, and return electrode 5 emerging from the insulation of a blade. This arrangement in which electrodes have at least one region exposed without insulation is the preferred embodiment for either connecting blades to handles or to have blades connect to other features, such as shafts, that will become part of a final device. The exposed regions of the electrodes can vary and the lengths of the blades can be stepped or otherwise made unique to facilitate producing electrical contact surfaces.

From an overall system standpoint, the DC power source could be part of the ESU or may be external to the ESU. When external to the ESU the DC power source could be an adapter connected to the ESU to which a surgical instrument is connected or the DC power source could be a part of the surgical instrument. The DC power source could be self contained, such as a battery, could obtain power from an outside source, such as an AC wall outlet, or it could obtain its power from the RF power supplied to the instrument by the ESU. When obtaining power from the RF power supplied by the ESU one or more rectifying components such as diodes would be used. Typically one or more electronic components, such as capacitors, would be used to isolate the ESU from the DC power being added to the RF power supplied to the instrument while still allowing RF power to be conveyed to the instrument.

Which electrodes are active, return, and passive may be fixed and unchanging or the polarities of the electrodes may change during use. Changing polarities during use may facilitate procedures such as making incisions by reducing the amount of force required to move a blade through tissue. Switches would be used to change the connections of the electrodes to active, return, and DC power poles. Typically such switches would use one or more electronic semiconductor components such as bipolar transistors, field effect transistors, or insulated gate bipolar transistors. The switching can be facilitated by timing the transition from one polarity setting to another during those times when the RF voltage applied to the blade is substantially less than the peak voltages applied by the ESU. Such low voltage switching would include switching during the times when voltages are close to zero, such as commonly occur with ESU outputs having crest factors greater than about 1.5, and commonly are greater than 2 or when ESU outputs have duty cycles less than 100% and commonly less than 75 percent.

Closely spaced electrodes may be made by placing a thin coat of an insulating material that bonds to electrode material on an individual electrode element and then placing another electrode element on the insulating bonding material. The bonding material needs to produce a surface with dielectric strength suitable for withstanding the voltage difference across the electrodes. Suitable materials include polydiorganosiloxanes, silicone elastomers, fluorosilicones, and polytetrafluoroethylenes. Other approaches include laminating a solid polymer sheet between electrode elements and interposing layers of adhesive. Additional approaches include using ceramic material that bonds the electrodes, including formulating the ceramic with particles or fibers with dimensions that space the electrodes apart to facilitate maintaining desired electrode spacing and planarity. The preferred approach is to use a ceramic material to bond the electrodes such that the bonding material extends between the electrodes to the exposed surfaces of the electrodes. The preferred ceramic material to use for bonding is one of the outer insulating materials described below. Even more preferred is to use one of the insulating materials described below that includes one or more hydrolyzable silanes including those that have halogens and even more preferable is to use one of the insulating materials described below that contain one or more hydrolyzable silanes that contain fluorine.

Preferably, the bonding material used between electrodes has added to it particles that are not electrically conductive that will space the electrodes apart when the electrodes are pressed together or otherwise fixtured during manufacturing. Examples of such particles are glass beads or fibers, ceramic beads or fibers, or polymeric beads or fibers. Preferably, such particles are generally rigid and capable of withstanding temperatures greater than 200° F. without deforming under load, such as glass or ceramic beads or fibers. Even more preferably, such spacing particles individually have approximately uniform dimensions such as being spherical. The spacing particles can comprise a range of dimensions, but in general the largest size particles will be the ones that hold the electrodes apart when they are pressed together or otherwise fixtured. The maximum diameter of the largest particles, or equivalent dimension that determines the spacing of the electrodes, is preferably between about 0.001 and 0.2 inches, and more preferably between about 0.002 and 0.100 inches and most preferably between about 0.005 and 0.015 inches.

The electrodes may be partially insulated from contact with tissue or with electrically conductive substances in contact with tissue by surrounding part of the blade with insulation. The insulation may be used to reduce the transmission of both electrical and thermal energy from the blade. Typically only the surfaces of the blade that produce the predetermined surgical effect, called the functional surfaces, would be left uninsulated. All surfaces that are not functional are called non-functional. For example, the leading edges of the electrodes in a blade being used for incisions would be the functional surfaces and they would be left exposed while the sides and backs of the electrodes would be insulated because they are non-functional. Insulating non-functional surfaces reduces the amount of DC power that needs to be supplied to reduce or eliminate deposition of ETDPs as well as reducing the amount of RF power that needs to be supplied to achieve the predetermined surgical effect.

In one aspect of the present invention, the outer insulating layer may be advantageously provided to have a maximum thermal conductance of about 1.2 W/cm²-° K when measured at about 300 ° K, more preferably about 0.12 W/cm²-° K or less when measured at about 300° K, and most preferably about 0.03 W/cm²-° K when measured at about 300 ° K. For purposes hereof, thermal conductance is intended to be a measure of the overall thermal transfer across any given cross section (e.g. of the insulation layer), taking into account both the thermal conductivity of the materials comprising such layer and the thickness of the layer (i.e. thermal conductance of layer=thermal conductivity of material comprising the layer (W/cm ° K)/thickness of the layer (cm)). In relation to the foregoing aspect, the insulation layer should also exhibit a dielectric withstand voltage of at least the peak-to-peak voltages that may be experienced by the electrosurgical instrument during surgical procedures. The peak voltages will depend upon the settings of the RF source employed, as may be selected by clinicians for particular surgical procedures. For purposes of the present invention, the insulation layer should exhibit a dielectric withstand voltage of at least about 50 volts, and more preferably, at least about 150 volts. As employed herein, the term dielectric withstand voltage means the capability to avoid an electrical breakdown (e.g. an electrical discharge through the insulating layer).

In one embodiment, the insulating layer may comprise a porous ceramic material that has had at least the pores on the surface sealed to prevent or impede the penetration of biological materials into the pores. Said ceramic may be applied to the electrodes via dipping, spraying, etc, then cured via drying, firing, etc. Preferably, the ceramic insulating layer should be able to withstand temperatures of at least about 2000° F. The ceramic insulating layer may comprise various metal/non-metal combinations, including for example compositions that comprise the following: aluminum oxides (e.g. alumina and Al₂O₃), zirconium oxides (e.g. Zr₂O₃), zirconium nitrides (e.g. ZrN), zirconium carbides (e.g. ZrC), boron carbides (e.g. B₄C), silicon oxides (e.g. SiO₂), mica, magnesium-zirconium oxides (e.g. (Mg—Zr)O₃), zirconium-silicon oxides (e.g. (Zr—Si)O₂), titanium oxides (e.g., TiO₂) tantalum oxides (e.g. Ta₂O₅), tantalum nitrides (e.g. TaN), tantalum carbides (e.g., TaC), silicon nitrides (e.g. Si₃N₄), silicon carbides (e.g. SiC), tungsten carbides (e.g. WC) titanium nitrides (e.g. TiN), titanium carbides (e.g., TiC), nibobium nitrides (e.g. NbN), niobium carbides (e.g. NbC), vanadium nitrides (e.g. VN), vanadium carbides (e.g. VC), and hydroxyapatite (e.g. substances containing compounds such as 3Ca₃ (PO₄)₂ Ca(OH)₂ Ca10(PO₄)₆ (OH)₂ Ca5(OH)(PO₄)₃, and Ca10H₂ O₂6 P₆). One or more ceramic layers may be employed, wherein one or more layers may be porous, such as holes filled with one or more gases or vapors. Such porous compositions will usually have lower thermal conductivity than the nonporous materials. An example of such materials are foam e.g., an open cell silicon carbide foam. Such porous materials have the disadvantage that they allow fluids, vapors, or solids to enter the pores whereby they are exposed to prolonged contact with high temperatures which can lead to thermal decomposition or oxidation and produce smoke or other noxious or possibly dangerous materials. Sealing the surface of the ceramic prevents such incursions, while substantially preserving the beneficial reduced thermal conductivity of the pores.

Ceramic coatings or electrode bonding materials may also be formed in whole or part from preceramic polymers that when heated form materials containing Si—O bonds able to resist decomposition when exposed to temperatures in excess of 1200° F., including compositions that use one or more of the following as preceramic polymers: silazanes, polysilzanes, polyalkoxysilanes, polyureasilazane, diorganosilanes, polydiorganosilanes, silanes, polysilanes, silanols, siloxanes, polysiloxanes, silsesquioxanes, polymethylsilsesquioxane, polyphenyl-propylsilsesquioxane, polyphenylsilsesquioxane, polyphenyl-vinylsilsesquioxane. Preceramic polymers may be used to form the ceramic coating by themselves or with the addition of inorganic fillers such as clays or fibers, including those that contain silicon oxide, aluminum oxides, magnesium oxides, titanium oxides, chrome oxides, calcium oxides, or zirconium oxides.

Ceramic coatings may also be formed by mixing one or more colloidal silicate solutions with one or more filler materials such as one or more fibers or clays. Preferably, the filler materials contain one or more materials that have at least 30 percent by weight Al₂O₃ or SiO₂ either alone or combined with other elements, such occurs in kaolin or talc. The colloidal silicate and filler mixture may optionally contain other substances to improve adhesion to electrode surfaces or promote producing a sealed or hydrophobic surface. Representative examples of colloidal silicate solutions are alkali metal silicates, including those of lithium polysilicate, sodium silicate and potassium silicate, and colloidal silica. Fibers that include those that contain in part or wholly alumina or silica or calcium silicate, and Wollastonite. Clays include those substances that are members of the smectite group of phyllosilicate minerals. Representative examples of clay minerals include bentonite, talc, kaolin (kaolinite), mica, clay, sericite, hectorite, montmorillonite and smectite. In the present invention, there are preferably used at least one of kaolin, talc, and montmorillonite. These clay minerals can be used singly or in combination. Preferably, at least one dimension, such as diameter or particle size of at least one of the filler materials has a mean value of less than 200 micrometers and more preferably has a mean value of less than 50 micrometers and even more preferably has a mean value of less than 10 microns and still more preferably has a mean value less than 5 microns. Substances that may be added to promote adhesion or production of a sealed or hydrophobic surface include those that increase the pH of the mixture, including sodium hydroxide or potassium hydroxide, and hydrolyzable silanes that condense to form one or more cross-linked silicone-oxygen-silicon structures.

Sealing the porous insulator is accomplished not by coating the ceramic in the sense that electrosurgical accessories have been coated with PTFE, silicone polymers and other such materials. Best surgical performance occurs when accessories are thin, therefore pores are best filled by a material that penetrates the surface of the porous material and seals the pores. Some residual material may remain on the surface, but such material is incidental to the sealing process.

Sealing materials need to withstand temperatures exceeding 400° F. and more preferably withstand temperatures exceeding 600° F. Silicates and solutions containing or forming silicates upon curing are the preferred materials. Other materials may be used, including silicone and fluorosilicones. For sealing the materials need to have low viscosity and other properties that enable penetration into the surface of the porous insulator. Traditional silicone and fluorosilicone polymer-forming compounds do not have these properties unless they are extensively diluted with a thinning agent, such as xylene or acetone.

The sealed porous insulation may be advantageously employed to yield an average maximum thermal conductivity of about 0.006 W/cm-° K or less where measured at 300° K. The insulating layer may preferably have a thickness of between about 0.001 and 0.2 inches, and most preferably between about 0.005 and 0.100 inches and most preferably between about 0.005 and 0.050 inches.

A coating that is applied as a single substance that upon curing does not require sealing may also be used for the outer insulation or as the bonding material between electrodes. Examples of such coatings formed from mixtures that use one or more of the aforementioned colloidal silicates and clays and also use one or more substances that reduce the surface free energy of the surface. Such substances that reduce the surface free energy include halogenated compounds, preferably, fluoropolymer compounds, such as PTFE and PFA, including aqueous dispersions of such compounds, organofunctional hydrolyzable silanes, including those containing one or more fluorine atoms on one or more pendant carbon chains.

Most preferably a hydrolyzable silane is a component in the coating or in the insulating material between electrodes with the hydrolyzable silane having one or more halogen atoms and having a general formula of CF₃(CF₂)ₘ(CH₂)nSi(OCH₂CH₃)₃ where m is preferably less about 20 and more preferably about 5 or less and where n is preferably about 2. Other groups besides (OCH₂CH₃)₃, such as those based on ethyl groups, may be substituted may be used and fall within the new art of this patent when they also are hydrolyzable. Other halogens, such as chlorine, may be substituted for the fluorine, although these will typically produce inferior results.

Preferably, the surface free energy (also referred to as the surface tension) of the coating is less than about 32 millinewtons/meter and more preferably less than about 25 millinewtons/meter and even more preferably less than about 15 millinewtons/meter and yet more preferably less than about 10 millinewtons/meter.

In another aspect of the present invention, the electrodes of the inventive electrosurgical instrument may be provided to have a thermal conductivity of at least about 0.35 W/cm ° K when measured at about 300° K. By way of primary example, the electrodes may advantageously comprise at least one metal selected from a group comprising: silver, copper, aluminum, gold, tungsten, tantalum, columbium (i.e., niobium), and molybdenum. Alloys comprising at least about 50% (by weight) of such metals may be employed, and even more preferably at least about 90% (by weight). Additional metals that may be employed in such alloys include zinc.

In yet another aspect of the present invention, at least a portion of the peripheral edge portion of the electrodes is not insulated (i.e. not covered by the outer insulating layer). In connection therewith, when the outer peripheral edge portion comprises copper such portion may be coated (e.g. about 10 microns or less) with a biocompatible metal. By way of example, such biocompatible metal may be selected from the group comprising: nickel, silver, gold, chrome, titanium tungsten, tantalum, columbium (i.e., niobium), and molybdenum.

In an additional aspect of the invention, it has also been determined that a laterally tapered, or sharpened, uninsulated peripheral edge portion having a maximum cross-sectional thickness which is about 1/10 of the maximum cross-sectional thickness of the main body portion is particularly effective for achieving localized electrosurgical signal delivery to a tissue site. In the later regard, it has also been determined preferable that the outer extreme of the peripheral edge portion of the electrodes have a thickness of about 0.001 inches or less.

In an additional related aspect of the present invention, the electrodes may comprise two or more layers of different materials. More particularly, at least a first metal layer may be provided to define an exposed peripheral edge portion of the electrodes that is functional to convey an electrosurgical signal to tissue as described above. Preferably, such first metal layer may comprise a metal having a melting temperature greater than about 2600° F., more preferably greater than about 3000° F., and even more preferably greater than about 4000° F., thereby enhancing the maintenance of a desired peripheral edge thickness during use (e.g. the outer extreme edge noted above). Further, the first metal layer may preferably have a thermal conductivity of at least about 0.35 W/cm ° K when measured at 300° K.

For living human/animal applications, the first metal layer may comprise a first material selected from a group consisting of tungsten, tantalum, columbium (i.e., niobium), and molybdenum. All of these metals have thermal conductivities within the range of about 0.5 to 1.65 W/cm ° K when measured at 300° K. Preferably, alloys comprising at least about 50% by weight of at least one of the noted first materials may be employed, and even more preferably at least about 90% by weight.

In addition to the first metal layer the electrodes may further comprise at least one second metal layer on the top and/or bottom of the first metal layer. Preferably, a first metal layer as noted above is provided in a laminate arrangement between top and bottom second metal layers. To provide for rapid heat removal, the second metal layer(s) preferably has a thermal conductivity of at least about 2 W/cm ° K. By way of primary example, the second layer(s) may advantageously comprise a second material selected from a group consisting of copper, gold, silver and aluminum. Preferably, alloys comprising at least about 50% of such materials may be employed, and even more preferably at least about 90% by weight. It is also preferable that the thickness of the first metal layer and of each second metal layer (e.g. for each of a top and bottom layer) be defined at between about 0.001 and 0.25 inches, and even more preferably between about 0.005 and 0.1 inches.

One or more of the electrodes may be plated with gold or silver or alloys thereof to confer added oxidation resistance to the portions of the electrodes exposed to tissue or current flow or both. Such plating may applied using electroplating, roll-bonding or other means either after assembly or prior to assembly of the electrodes to form blades. The preferred plating thickness is a least about 0.5 micrometers and more preferably at least about 1 micrometer.

As may be appreciated, multi-layered metal bodies of the type described above may be formed using a variety of methods. By way of example, sheets of the first and second materials may be role-bonded together then cut to size. Further, processes that employ heat or combinations of heat and pressure may also be utilized to yield laminated electrodes.

In a further aspect of the present invention, the inventive electrosurgical instrument may further comprise a heat sink for removing thermal energy from the electrodes. In this regard, the provision of a heat sink establishes a thermal gradient away from the peripheral edge of the electrodes, thereby reducing undesired thermal transfer to a tissue site. More particularly, it is preferable for the heat sink to operate so as to maintain the maximum temperature on the outside surface of the insulating layer at about 160° C. or less, more preferably at about 80° C. or less, and most preferably at 60° C. or less. Relatedly, it is preferable for the heat sink to operate to maintain an average electrodes temperature of about 500° C. or less, more preferably of about 200° C. or less, and most preferable of about 100° C. or less.

In one approach, the heat sink may comprise a vessel comprising a phase change material that either directly contacts a portion of the electrodes (e.g. a support shaft portion) or that contacts a metal interface provided on the vessel which is in turn in direct contact with a portion of the electrodes (e.g. a support shaft portion). Such phase change material changes from a first phase to a second phase upon absorption of thermal energy from the electrodes. In this regard, the phase change temperature for the material selected should preferably be greater than the room temperature at the operating environment and sufficiently great as to not change other than as a consequence of thermal heating of the electrosurgical instrument during use. Such phase change temperature should preferably be greater than about 30° C. and most preferably at least about 40° C. Further, the phase change temperature should be less than about 225° C. Most preferably, the phase change temperature should be less than about 85° C.

The phase change may be either from solid to liquid (i.e., the phase change is melting) or from liquid to vapor (i.e., the phase change is vaporization) or from solid to vapor (i.e., the phase change is sublimation). The most practical phase changes to employ are melting and vaporization. By way of example, such phase change material may comprise a material that is an organic substance (e.g., fatty acids such as stearic acid, hydrocarbons such as paraffins) or an inorganic substance (e.g., water and water compounds containing sodium, such as, sodium silicate (2-)-5-water, sodium sulfate-10-water).

In another approach, the heat sink may comprise a gas flow stream that passes in direct contact with at least a portion of the electrodes. Such portion may be a peripheral edge portion and/or a shaft portion of the electrodes that is designed for supportive interface with a holder for hand-held use. Alternatively, such portion may be interior to at least a portion of the electrodes, such as interior to the exposed peripheral edge portion and/or the shaft portion of the electrodes that is designed for supportive interface with a holder for hand-held use. In yet other approaches, the heat sink may simply comprise a thermal mass (e.g. disposed in a holder).

In one arrangement of the present invention, an electrosurgical instrument comprises a main body portion having a blade-like configuration at a first end and an integral, cylindrical shaft at a second end. The main body may comprise a highly-conductive metal and/or multiple metal layers as noted. At least a portion of the flattened blade end of the main body is coated with a ceramic-based and/or silicon-based, polymer insulating layer, except for the peripheral edge portion thereof. The cylindrical shaft of the main body is designed to fit within an outer holder that is adapted for hand-held use by medical personnel. Such holder may also include a chamber comprising a phase-change material or other heat sink as noted hereinabove. Additionally, electrical, push-button controls may be incorporated into the holder for selectively controlling the application of one or more, predetermined, electrosurgical signal(s) from an RF energy source to the flattened blade via the shaft of the main body portion.

In the latter regard, conventional electrosurgical signals may be advantageously employed in combination with one or more of the above-noted electrosurgical instrument features. In particular, the inventive electrosurgical instrument yields particular benefits when employed with electrosurgical signals and associated apparatus of the type described in U.S. Pat. No. 6,074,387, hereby incorporated by reference in its entirety.

1. An electrosurgical system, comprising:
an AC power supply;
a DC power supply; and,
at least one active electrode and at least one return electrode electrically isolated from each other, wherein said at least one active electrode and said at least one return electrode are electrically interconnected to different poles of said AC power supply to define a bipolar relationship, and wherein at least one of said at least one active electrode and said at least one return electrode is electrically interconnected to a pole of said DC power supply.

2. An electrosurgical system as recited in claim 1, wherein said at least one active electrode and said at least one return electrode are electrically interconnected to different poles of said DC power supply.

3. An electrosurgical system as recited in claim 2, wherein a non-zero root mean square (RMS) voltage is establishable across said at least one active electrode and said at least one return electrode.

4. An electrosurgical system as recited in claim 3, wherein said DC power supply comprises:
a rectifying element having a reverse recovery time less than a period of the AC power supply.

5. An electrosurgical system as recited in claim 1, further comprising:
at least one passive electrode isolated from direct electrical interconnection with said AC power supply and electrically interconnected to a pole of said DC power supply.

6. An electrosurgical system as recited in claim 5, wherein at least one of said at least one active electrode and said at least one return electrode is electrically interconnected to a different pole of said DC power supply than said at least one passive electrode.

7. An electrosurgical system as recited in claim 6, wherein said at least one active electrode and said at least one return electrode are electrically interconnected to a common pole of said DC power supply.

8. An electrosurgical system as recited in claim 7, wherein said at least one passive electrode is electrically interconnected to a positive pole of DC power supply, and wherein both said at least one active electrode and said at least one return electrode are electrically interconnected to a negative pole of said DC power supply.

9. An electrosurgical system as recited in claim 7, further comprising:
at least one AC blocking component interposed in series between said DC power supply and one of said at least one active electrode and said at least one return electrode.

10. An electrosurgical system as recited in claim 9, wherein said at least one AC blocking component comprises:
an inductor adapted to provide at least about 500 ohms impedance in relation to an output frequency range of said AC power supply.

11. An electrosurgical system as recited in claim 7, further comprising:
at least one DC blocking component interposed in series between said return electrode and said AC power supply.

12. An electrosurgical system as recited in claim 7, further comprising:
at least one DC blocking component interposed in series between said AC power supply and said active electrode.

13. An electrosurgical system as recited in claim 12, wherein said at least one DC blocking component comprises:
a capacitor having an impedance of less than about 500 ohms in relation to an output frequency range of said AC power supply.

14. An electrosurgical system as recited in claim 5, wherein said DC power supply establishes a DC voltage difference between said at least one passive electrode and at least one of said at least one active electrode and said at least one return electrode of between about 0.5 volts and 100 volts.

15. An electrosurgical system as recited in claim 1, wherein said DC power supply is separate from said AC power supply.

16. An electrosurgical system as recited in claim 15, wherein said DC power supply comprises:
at least one battery.

17. An electrosurgical system as recited in claim 1, wherein said DC power supply comprises:
at least one active component, interconnected to said AC power supply, for converting an AC output from said AC power supply into a DC output.

18. An electrosurgical system as recited in claim 17, wherein said at least one active component comprises:
a plurality of rectifying elements configured in a bridge configuration.

19. An electrosurgical system as recited in claim 5, further comprising:
electrical insulating material interposed and interconnected between said at least one active electrode and said at least one return electrode, wherein a bipolar, electrosurgical blade is defined.

20. An electrosurgical system as recited in claim 19, wherein said electrical insulating material has a dielectric withstand strength of at least about 50 volts.

21. An electrosurgical system as recited in claim 19, wherein said passive electrode is separate from said electrosurgical blade.

22. An electrosurgical system as recited in claim 5, further comprising:
electrical insulating material interposed and interconnected between said at least one active electrode, said at least one return electrode and said at least one passive electrode, wherein a bipolar, electrosurgical blade is defined.

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)
